(19) United States
(12) Patent Application Publication
(10) Pub. No.: US 2022/0370506 A1
(43) Pub. Date: Nov. 24, 2022

(54) AUGMENTATION OF FIBROBLAST MEDIATED REGENERATION OF INTRAVERTEBRAL DISCS

(71) Applicant: FIGENE, LLC, Houston, TX (US)

(72) Inventors: Pete O'HEERON, Houston, TX (US); Thomas ICHIM, San Diego, CA (US)

(21) Appl. No.: 17/757,314

(22) PCT Filed: Dec. 28, 2020
(86) PCT No.: PCT/US20/67225

§ 371 Date: Jun. 14, 2022

Related U.S. Application Data
(60) Provisional application No. 62/953,841, filed on Dec. 26, 2019.

(51) Int. Cl. A61K 35/33; A61K 31/4045; A61K 35/12; A61K 9/127

(52) U.S. Cl. CPC A61K 35/33; A61K 31/4045; A61K 35/12; A61K 9/127

(57) ABSTRACT

Embodiments of the disclosure include methods of increasing the efficacy of a fibroblast cell therapy for any medical condition, including degenerative disc disease, by providing at least one anti-inflammatory composition, exosomes and/or apoptotic bodies, stem cells, or a combination thereof; and administering the fibroblast cell therapy. The anti-inflammatory composition may comprise a composition that inhibits and/or reduces TNF-alpha, such as melatonin.

This application claims priority to U.S. Provisional Patent Application Ser. No. 62/953,841, filed Dec. 26, 2019, which is incorporated by reference herein in its entirety.

### TECHNICAL FIELD

Embodiments of the disclosure include at least the fields of cell biology, molecular biology, and medicine.

### BACKGROUND

It is well known that the intervertebral disc contains a jelly-like nucleus pulposus surrounded by a fibrous annulus fibrosus. In humans, the spine is composed of bony structures called vertebrae, separated by intervertebral discs (IVD). One of the main functions of the vertebrae is to provide structural support and protection for the spinal cord. Each vertebrae is comprised of a spinous process, a bony prominence behind the spinal cord, which shields the cord's nervous tissue on the back side, two bony protrusions on the sides called transverse processes, and a “body” in front of the spinal cord which provides a structural support for bearing weight. The average adult has 24 vertebrae, although at birth 33 are present, this is due to fusion during normal development. The vertebrae are divided by anatomical locations with 7 in the neck, also called the cervical vertebrae, 12 in the middle back, called the thoracic vertebrae, 5 in the lower back, called the lumbar vertebrae, and the sacrum, which is actually formed from five fused vertebrae. The tailbone, called the coccyx is made of three fused vertebrae. Of these, the lumbar vertebrae are the largest, in part since they are responsible for carrying the majority of body weight. Because of this, the lumbar area is associated with the highest level of degeneration and is believed causative for a wide variety of pain-inducing syndromes.

Under an axial load (the weight of the patient), the nucleus pulposus compresses and radially transfers that load to the annulus fibrosus. The laminated nature of the annulus fibrosus provides it with a high tensile strength and so allows it to expand radially in response to this transferred load. Additionally, it is accepted that in a healthy intervertebral disc, cells within the nucleus pulposus produce an extracellular matrix (ECM) containing a high percentage of proteoglycans. These proteoglycans contain sulfated functional groups that retain water, thereby providing the nucleus pulposus with its cushioning qualities. These nucleus pulposus cells may also secrete small amounts of cytokines as well as matrix metalloproteinases (MMPs). These cytokines and MMPs help regulate the metabolism of the nucleus pulposus cells. In some instances of degenerative disc disease (DDD), gradual degeneration of the intervertebral disc is caused by mechanical instabilities in other portions of the spine. In these instances, increased loads and pressures on the nucleus pulposus cause the cells within the disc (or invading macrophages) to emit larger than normal amounts of the above-mentioned cytokines. In other instances of DDD, genetic factors or apoptosis can also cause the cells within the nucleus pulposus to emit toxic amounts of these cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to eliminate waste may result in the accumulation of high levels of toxins that may cause nerve irritation and pain.

Currently, the primary therapies for IVD degeneration are surgical interventions in which degenerated discs are excised or fused with neighboring discs. Surgical therapies aim to alleviate pain and other symptoms of IVD degeneration, but do nothing to repair or regenerate diseased IVDs.

One approach for treating degenerated IVD cells and tissues is the use of cell-based therapies, in which living cells are administered to repair, replace, and/or remodel diseased tissues. Several recent studies have investigated the use of cell-based therapies for degenerative IVD conditions. For example, U.S. Pat. Nos. 6,352,557 and 6,340,369 teach harvesting live IVD cells from a patient, culturing the cells and transplanting them into an affected IVD. These approaches, while theoretically promising, have shown limited effectiveness in repairing degenerated IVDs and suffer from complications caused by immunological incompatibility between cell donors and recipients. An alternative cell-based therapeutic approach is the use of stem cells, which have the ability to divide and differentiate into cells comprising diseased tissues. Transplantation of stem cells can be utilized as a clinical tool for reconstituting a target tissue, thereby restoring physiologic and anatomic functionality. The application of stem cell technology is wide-ranging, including tissue engineering, gene therapy delivery, and cell therapeutics, i.e., delivery of biotherapeutic compositions to a target location via exogenously supplied living cells or cellular components that produce or contain those compositions. One obstacle to realization of the therapeutic potential of stem cell technology has been the difficulty of obtaining sufficient numbers of stem cells. Embryonic, or fetal tissue, is one source of stem cells. Embryonic stem and progenitor cells have been isolated from a number of mammalian species, including humans, and several such cell types have been shown capable of self-renewal and expansion, as well differentiation into a number of different cell lineages. However, the derivation of stem cells from embryonic and fetal sources has raised many ethical and moral issues that have prevented further development of embryonic stem cell therapeutics. Additionally, embryonic and fetal stem cells have been shown to cause and/or promote cancer. The current disclosure teaches means of overcoming current limitations of cellular therapy for IVD based on using fibroblasts and combinations of fibroblasts with compositions to enhance therapeutic activity of said fibroblasts. The disclosure further provides means of utilizing fibroblasts and manipulating fibroblasts as a means of circumventing the need for stem cells in the treatment of IVD.

### BRIEF SUMMARY

The present disclosure is directed to a system and method for improving the efficacy of one or more therapies, such as therapies for degenerative disc diseases. Certain embodiments of the disclosure concern increasing the efficacy of at least one cell therapy, such as at least one fibroblast cell therapy. In some embodiments, increasing the efficacy of the therapy(ies) comprises administering at least one anti-inflammatory composition, exosomes, apoptotic bodies, and/or stem cells to an individual in combination with administering a fibroblast cell therapy to the individual. In some embodiments, the anti-inflammatory composition inhibits TNF-alpha, reduces TNF-alpha, suppresses the responsiveness of cells to activation of TNF-alpha receptors, and/or inhibits responsiveness to TNF-alpha.

The composition that inhibit and/or reduce TNF-alpha may be selected from the group consisting of etanercerpt, methylprednisolone, cycloheximide, auranofin, sodium aurothiomalate, triethyl gold phosphine Ethanol, leukotriene B4, interleukin-4, interleukin-13, polymyxin B, bile acids, interleukin-6, lactulose, oxpentifylline, mometasone, glucocorticoids, colchicine, chloroquine, FK-506, berberine, resveratrol, pterostilbene, vitamin A, vitamin C, cyclosporine, phosphodiesterase inhibitors such as vinpocetine, milrinone, CI-930, rolipram, nitroquazone, zaprinast, synthetic lipid A, amrinone, N-acetylcysteine, dithiocarbamates and metal chelators, exosurf synthetic surfactant, dehydroepiandrosterone, delta-tetrahydrocannabinol, phosphatidylserine, TCV-309, PAF antagonist, thalidomide, cytochrome p450 inhibitors such as Metyrapone and SKF525A, cytochalasin D, ketamine, TGF-beta, interleukin-10, pentoxifylline, BRL 61,063, calcium antagonists such as dantrolene, azumolene, and diltiazem, curcumin, kappa-selective opioid agoinst U50,488H (trans-3,4-dichloro-N-methyl-N-[7-(1-pyrrolidinyl)cyclohexyl]benzene-acetamide methanesulfonate), alendronate, alkaloids such as fangchinoline and isotetrandrine, plant alkaloids such as tetrandrine, sulfasalazine, epinephrine, BMS-182123, adenosine, E3330, nicotine, IVIG, cardiotrophin-1, KB-R7785, CGRP, ligustrazine, dexanabinol, iloprost, activated protein C, growth hormone, spermine, FR-167653, gm-6001, estradiol, aspirin, amiodarone, melatonin, and a combination thereof.

The composition that inhibits responsiveness to TNF-alpha may be selected from the group consisting of ibuprofen, indomethacin, nedocromil sodium, cromolyn (sodium cromoglycate), spleen derived factors, pentoxifylline, the 30 kDa TNF-alpha inhibitor, NG-methyl-L-arginine, antibodies directed against the core/lipid A, dexamethasone, chlorpromazine, activated alpha 2 macroglobulin, serum amyloid A protein, neutrophil derived proteolytic enzymes, phentolamine, propranolol, leukotriene inhibitors, nordihydroguaiaretic acid, genistein, butylated hydroxyanisole, CNI-1493, quercetin, gabexate mesylate, SM-12502, monoclonal nonspecific suppressor factor (MNSF), pyrrolidine dithiocarbamate (PDTC), aprotinin, and a combination thereof.

In some embodiments, the fibroblasts comprising the one or more fibroblast therapies are cultured in a manner to adapt the fibroblasts to the intradiscal environment. In some embodiments, fibroblasts comprising the one or more fibroblast therapy are modified to prevent or reduce the expression or secretion of one or more inflammatory cytokines. The fibroblasts may be genetically engineered and/or exposed to compositions that prevent or reduce the expression or secretion of one or more inflammatory cytokines. In some embodiments, the fibroblasts are exposed to TNF-alpha to prevent or reduce the expression or secretion of one or more inflammatory cytokines. The inflammatory cytokines encompassed herein may be selected from the group consisting of interleukin-1, interleukin-6, interleukin-7, interleukin-9, interleukin-11, interleukin-12, interleukin-8, interleukin-15, interleukin-17, interleukin-18, interleukin-21, interleukin-23, interleukin-27, interleukin-33, interferon-gamma, TNF-alpha, HMGB-1 and a combination thereof.

The fibroblasts of the present disclosure may comprise fibroblasts selected from the group consisting of skin fibroblasts, hair follicle fibroblasts, adipose fibroblasts, bone marrow fibroblasts, umbilical cord blood fibroblasts, placental fibroblasts, omentum fibroblasts, ovarian tube fibroblasts, peripheral blood fibroblasts, and a combination thereof. In some embodiments, fibroblasts of the present disclosure are isolated from tissue comprising fibroblasts selected from the group consisting of skin fibroblasts, hair follicle fibroblasts, adipose fibroblasts, bone marrow fibroblasts, umbilical cord blood fibroblasts, placental fibroblasts, omentum fibroblasts, ovarian tube fibroblasts, peripheral blood fibroblasts, and a combination thereof.

In some embodiments, the compositions, including anti-inflammatory compositions, exosomes, apoptotic bodies, and/or stem cells, and/or therapies, including fibroblast cell therapies, encompassed herein are administered to the disc of an individual. The individual may have a degenerative disc disease. The disc may comprise nucleus pulposus. In some embodiments, the compositions, including anti-inflammatory compositions and/or exosomes, and/or therapies, including fibroblast and/or stem cell therapies, administered to the individual reduce inflammation in a disc in the individual. The inflammation may be associated with an aspect selected from the group consisting of: the ERK2 pathway, the JAK/STAT pathway, the NF-kappa B pathway, perispinal neutrophils, perispinal monocytes, inflammatory cytokines, and a combination thereof.

The inflammatory cytokines encompasses herein may include interleukin-1, interleukin-6, interleukin-7, interleukin-9, interleukin-11, interleukin-12, interleukin-8, interleukin-15, interleukin-17, interleukin-18, interleukin-21, interleukin-23, interleukin-27, interleukin-33, interferon-gamma, TNF-alpha, and a combination thereof. In some embodiments, the inflammatory cytokines induce STAT3 activation in cells of hematopoietic origin, which may include monocytes. In some embodiments, the inflammatory cytokines activate at least one inhibitor of kappa B (IkB) kinase. In some embodiments, the inflammatory cytokines activate NF-kappa B.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description

### DETAILED DESCRIPTION

### I. Degenerative Disc Disease

The disclosure provides means of augmenting therapeutic efficacy of cells implanted into the intervertebral disc for treatment of disc degenerative disease. In certain embodiments the disclosure, the efficacy of fibroblasts, for example as a therapeutic modality for treatment of disc degenerative disease, may be improved by modifying the environment of at least one intervertebral disc.

Methods encompassed herein concern the treatment of a degenerative disc disease. The degenerative disc disease may comprise any disease wherein at least one intervertebral disc in an individual has deteriorated or is in the process of deteriorating. The disc may comprise nucleus pulposus. The deterioration may be from any cause, including drying of the discs, physical activity, any injury of the disc, swelling, inflammation or a combination thereof. The one or more discs in the individual may be thinning or may comprise bone spurs. The one or more discs may be herniated, bulged, slipped, and/or ruptured. The individual may or may not have back pain due to the degeneration of the one or more discs.

In some embodiments, the degenerative disc disease comprises inflammation in at least one disc in the individual. The inflammation may be caused by, an aspect, such as activation, of the ERK2 pathway, the JAK/STAT pathway, the NF-kappa-B pathway, or a combination thereof. The inflammation may be caused by perispinal monocytes and/or inflammatory cytokines. In some embodiments, the inflammatory cytokines induce STAT3 activation of hematopoietic origin, including monocytes. In some embodiments, the inflammatory cytokines activate at least one inhibitor of kappa B kinase (IkB kinase), NF-kappa-B, or a combination thereof. The inflammatory cytokines encompassed herein may include interleukin-1, interleukin-6, interleukin-7, interleukin-9, interleukin-11, interleukin-12, interleukin-8, interleukin-15, interleukin-17, interleukin-18, interleukin-21, interleukin-23, interleukin-27, interleukin-33, interferon-gamma, TNF-alpha, or a combination thereof.

### II. Anti-Inflammatory Compositions

The disclosure concerns, intra alia, the reduction of underlying inflammation before, concurrent with, and/or subsequent to administration of cellular therapies, such as fibroblasts. Reduction of disc inflammation may be accomplished by several means according to the current disclosure. In certain embodiments, inflammation is reduced by administration of one or more antioxidants, and/or anti-inflammatory compositions. In some embodiments, anti-inflammatory compositions comprise compositions that inhibit TNF-alpha, reduce TNF-alpha, suppresses the responsiveness of cells to activation of TNF-alpha receptors, and/or inhibit the response to TNF-alpha. In some cases the anti-inflammatory composition may be an antibody against TNF alpha. Such antibodies are clinically available an commonly used for treatment of rheumatoid arthritis.

In certain embodiments, the anti-inflammatory composition is a fusion protein, such as etanercept. Administration of at least one fusion protein, including etanercept, may be performed prior to, concurrent with and/or subsequent to a cellular therapy, including any cellular therapy encompassed herein. Various doses for administration of etanercept are known to one of skill in the art. In certain embodiments, an individual, including any individual encompassed herein, is treated with subcutaneous etanercept at a dose of 25 mg on days 1, 4, and 7. In specific cases, the individual is treated with subcutaneous etanercept at a dose of 1, 5, 19, 15, 20, 25, 30, 35, 50, 75, or 100 mg on days 1, 2, 3, 4, 5, 6, 7, 8, 9, 10, or later. Other types of inflammatory and/or antioxidants may be added. For example, some patients may be given intravenous methylprednisolone at a dose of 250 mg on days 1, 4, and 7 as an addition to said etanercept. In specific cases, the individual is provided intravenous methylprednisolone at a dose of 100, 200, 225, 250, 275, 300, 325, or greater mg on days 1, 2, 3, 4, 5, 6, 7, 8, 9, or 10 or greater as an addition to said etanercept.

The use of compositions that block TNF-alpha, such as etanercept, alone for treatment of lower back pain has not been shown to work effectively to reduce symptoms. For example, one study evaluated escalating doses of intradiscal etanercept in 36 patients with chronic lumbosacral radiculopathy or discogenic low back pain. A double-blind, placebo-controlled pilot study was conducted whereby six patients received 0.1, 0.25, 0.5, 0.75, 1.0, or 1.5 mg etanercept intradiscally in each pain-generating disc. In each escalating dose group of six patients, one received placebo. A neurologic examination and postprocedure leukocyte counts were performed in all patients at 1-month follow-up visits. In patients who experienced significant improvement in pain scores and function, follow-up visits were conducted 3 and 6 months after the procedure. At 1-month follow-up, no differences were found for pain scores or disability scores between or within groups for any dose range or subgroup of patients. Only eight patients remained in the study after 1 month and elected to forego further treatment. No complications were reported, and no differences were noted between preprocedure and postprocedure leukocyte counts. The authors concluded that although no serious side effects were observed in this small study, a single low dose of intradiscal etanercept does not seem to be an effective treatment for chronic radicular or discogenic low back pain [1]. Accordingly, although TNF blockade did not seem to be effective, such blockade may be useful for the purpose of protecting the integrity and composition of the nucleus pulposus such that when cells with regenerative properties, including cellular therapies encompassed herein, are administered, said cells enter “fertile ground” and will possess a higher propensity of inducing regeneration. Intradiscal administration of anti-TNF alpha antibodies has been described in other studies and appears to possess an acceptable safety profile [2-4]

In some embodiments, compositions capable of inhibiting inflammation are administered to an individual, such as interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric .acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

In some embodiments of the disclosure, inhibition of inflammation is accomplished as a means of preparing the disc for administration of at least one cellular therapy, such as at least one fibroblasts and/or stem cell therapy including those encompassed herein. Inhibition of inflammation may be accomplished by administration of inhibitors of TNF-alpha production and/or inhibitors of TNF-alpha signaling. Compositions known to inhibit TNF-alpha production and/or responsiveness to TNF-alpha, and/or inflammation may be selected from the group consisting of cycloheximide [5], auranofin, sodium aurothiomalate, and triethyl gold phosphine [6], lipoxygenase inhibitors [7-10], ethanol [11, 12], Leukotriene B4 [13], interleukin-4 [14], interleukin-13 [15], polymyxin B [16, 17], bile acids [18], interleukin-6 [19], lactulose [20], oxpentifylline [21], mometasone [22], glucocorticoids [23], colchicine [24], chloroquine [25], FK-506 [26, 27], cyclosporine [28], phosphodiesterase inhibitors such as vinpocetine, milrinone, CI-930, rolipram, nitroquazone, zaprinast [29], synthetic lipid A [30, 31], amrinone [32], N-acetylcysteine [33], dithiocarbamates and metal chelators [34], exosurf synthetic surfactant [35], dehydroepiandrosterone [36], delta-tetrahydrocannabinol [37, 38], phosphatidylserine [39], TCV-309, a PAF antagonist [40], thalidomide [41-43], cytochrome p450 inhibitors such as Metyrapone and SKF525A [44], cytochalasin D [45], ketamine [46], TGF-beta [47], interleukin-10 [48], pentoxifylline [49], BRL 61,063 [50], calcium antagonists such as dantrolene, azumolene, and diltiazem [51], curcumin [52], kappa-selective opioid agoinst U50,488H (trans-3,4-dichloro-N-methyl-N-[7-(1-pyrrolidinyl)cyclohexyl]benzene-acetamide methanesulfonate) [53], alendronate [54], alkaloids such as fangchinoline and isotetrandrine [55], plant alkaloids such as tetrandrine [56], sulfasalazine [57], epinephrine [58], BMS-182123 [59], adenosine [60, 61], E3330 [62], nicotine [63, 64], WIG [65, 66], cardiotrophin-1 [67], KB-R7785 [67], CGRP [68], ligustrazine [69], dexanabinol [70], iloprost [71], activated protein C [72], growth hormone [73], spermine [74], FR-167653 [75], gm-6001 [76], estradiol [77], aspirin [78], amiodarone [79], melatonin, and a combination thereof.

In some embodiments, inhibitors of the effects of TNF-alpha production are administered either systemically and/or intradiscally to suppress inflammation and allow for enhancement of therapeutic effects of cells and/or regenerative factors (such as VEGF, PDGF-bb, IGF, NGF, FGF-1, FGF-2, FGF-5, EGF, and CTNF administered intradiscally. Some examples of compositions which inhibit activities of TNF-alpha include ibuprofen and indomethacin [80], Nedocromil sodium and cromolyn (sodium cromoglycate) [81], spleen derived factors [82], pentoxifylline [83-85], the 30 kDa TNF-alpha inhibitor [86], NG-methyl-L-arginine [87], antibodies directed against the core/lipid A [88], dexamethasone [89], chlorpromazine [90], activated alpha 2 macroglobulin [91], serum amyloid A protein [92], neutrophil derived proteolytic enzymes [93], phentolamine and propranolol [94], leukotriene inhibitors [95], nordihydroguaiaretic acid [96], genistein [97], butylated hydroxyanisole [98], CNI-1493 [99], quercetin [100], gabexate mesylate [101], SM-12502 [102], monoclonal nonspecific suppressor factor (MNSF) [103], pyrrolidine dithiocarbamate (PDTC) [104], and aprotinin [105].

Anti-inflammatory compositions encompassed herein may be administered to an individual, including any individual encompassed herein. One or more compositions may be administered to the individual systemically and/or locally. One or more compositions may be administered to the individual intravenously, intradiscally, epidurally, intrarectally, intra-omental, or a combination thereof. The anti-inflammatory composition may be administered prior to or simultaneously with the compositions and/or cellular therapies encompassed herein.

### III. Exosomes and Apoptotic Bodies

In some embodiments of the disclosure, exosomes are administered to an individual, including any individual encompassed herein, as a means of enhancing ability of at least one cellular therapy, including at least one fibroblast therapy, to engraft intradiscally and induce regeneration. Exosomes may be purified from a variety of sources, such as from fibroblasts, platelets, M2 macrophages, mesenchymal stem cells, or a combination thereof.

In some embodiments, apoptotic bodies of cells are utilized to enhance the activity and/or engraftment of at least one cellular therapy, including at least one fibroblast therapy. Apoptotic bodies may be generated from any cell undergoing apoptosis. Apoptosis may be induced in vitro using known methods in the art, including treatment with photosensitizers followed by irradiation, gamma irradiation, X-radiation; induction of mitotic arrest; and/or exposure to ozone gas. Apoptosis-characterizing features may include, but are not limited to, surface exposure of phosphatidylserine, as detected by standard, accepted methods of detection such as Annexin V staining; alterations in mitochondrial membrane permeability measured by standard methods; DNA fragmentation as measured by accepted methods of analyzing DNA fragmentation such as the appearance of DNA laddering on agarose gel electrophoresis following extraction of DNA from the cells or by in situ labeling. In some embodiments, apoptotic bodies, such as allogeneic fibroblast apoptotic bodies, may be administered alone, or may be administered together with a tolerogenic adjuvant such as immature dendritic cells, T regulatory cells, mesenchymal stem cells, fibroblasts, and/or gene modified cells.

Methods of the present disclosure may include the incubation of fibroblasts with added factors that further enhance generation or function of the T regs. Suitable factors include, without limitation, hormones, proteins, drugs, and/or antibodies. The factors may include, without limitation, at least one of TGF-β, α-MSH, anti-CD46, IL-10, vitamin D3, dexamethasone, rapamycin, and/or IL-2. The factor may be IL-10. The IL-10 may be present at a concentration of about 1 ng/mL to about 100 ng/mL. The IL-10 may be present at a concentration of about 20 ng/mL.

In some embodiments, ECP is used to induce apoptosis. This may involve a photoactivatable composition added to a cell population ex vivo. The photosensitive (synonymous with photoactivatable) composition may be administered to a cell population comprising blood cells following withdrawal from an individual, subject, recipient, and/or donor, as the case may be, and prior to or contemporaneously with exposure to ultraviolet light. The photosensitive composition may be administered to a cell population comprising whole blood or a fraction thereof, provided that the target blood cells or blood components receive the photosensitive composition. In one embodiment, fibroblasts allogenic to an individual receiving a cellular therapy are mixed with autologous blood from the individual and the combination of allogenic fibroblasts and autologous blood is subjected to photopheresis. In another embodiment, a portion of the individual's blood, recipient's blood, or donor's blood could first be processed using known methods to substantially remove erythrocytes; the photoactive composition may then be administered to the resulting cell population comprising the enriched PBMC fraction.

Photoactivatable compositions for use in accordance with the present disclosure include, but are not limited to, psoralens (or furocoumarins) as well as psoralen derivatives such as those described in, for example, U.S. Pat. Nos. 4,321,919; and 5,399,719. Photoactivatable compositions include 8-methoxypsoralen; 4,5′8-trimethylpsoralen; 5-methoxypsoralen; 4-methyl psoralen; 4,4-dimethylpsoralen; 4-5′-dimethylpsoralen; 4′-aminomethyl-4,5′,8-trimethylpsoralen; 4′-hydroxymethyl-4,5′,8-trimethylpsoralen; 4′,8-methoxypsoralen; and a 4′-(omega-amino-2-oxa) alkyl-4,5′8-trimethylpsoralen, including but not limited to 4′-(4-amino-2-oxa)butyl-4,5′,8-trimethylpsoralen. In one embodiment, one or more photosensitive compositions that may be used comprise the psoralen derivative, amotosalen (S-59) (Cerus Corp., Concord, Calif.). In certain embodiments, one or more photosensitive compositions comprise 8-methoxypsoralen (8 MOP). The cell population to which the photoactivatable composition has been added may be treated with a light of a wavelength that activates the photoactivatable composition. The treatment step that activates the photoactivatable composition is preferably carried out using long wavelength ultraviolet light (UVA), for example, at a wavelength within the range of 320 to 400 nm. The exposure to ultraviolet light during the photopheresis treatment preferably is administered for a sufficient length of time to deliver about 1-2 J/cm² to the cell population. Extracorporeal photopheresis apparatus useful in the methods according to the disclosure include those manufactured by Therakos, Inc., (Exton, Pa.) under the name UVAR®. A description of such an apparatus is found in U.S. Pat. No. 4,683,889. Treatment with apoptotic fibroblasts, in one embodiment of the disclosure is utilized to inhibit dendritic cell maturation, in part, through induction of blockade of NF-kappa B activation. In some embodiments of the disclosure, fibroblasts are transfected with autoantigens that are found in inflammatory bowel disease prior to generation of apoptotic bodies.

Exosomes and/or apoptotic bodies encompassed herein may be administered to an individual, including any individual encompassed herein. Exosomes and/or apoptotic bodies may be administered to the individual systemically and/or locally. Exosomes and/or apoptotic bodies may be administered to the individual intravenously, intradiscally, epidurally, intrarectally, intra-omentally, intra-arterially, or a combination thereof. Exosomes and/or apoptotic bodies may be administered prior to or simultaneously with the compositions and/or cellular therapies encompassed herein.

### IV. Stem Cells

In some embodiments, stem cells are administered to an individual, including any individual encompassed herein, to improve the efficacy of a fibroblasts cell therapy. The stem cells may modify the environment, including the environment in the disc, where the fibroblast cell therapy is administered to improve the efficacy of a fibroblast cell therapy. The stem cells may reduce inflammation in the environment, including the environment in the disc, where the fibroblast cell therapy is administered to improve the efficacy of a fibroblast cell therapy. Stem cells of the present disclosure may include mesenchymal stem cells, inducible pluripotent stem cells, stress induced stem cells, parthenogenic derived stem cells, embryonic stem cells, somatic cell nuclear transfer derived stem cells, hematopoietic stem cells, or derivatives thereof, for example.

Stem cells encompassed herein may be modified (such as with incubation with hypoxia) to reduce inflammation or otherwise modify an environment, including an environment in the disc, of an individual. In one embodiment, stem cells are incubated for a time period in conditions containing lower oxygen than normally found in atmospheric conditions. In one preferred embodiment cells are incubated for a period of 24-48 hours at conditions of 1% oxygen.

Stem cells encompassed herein may be administered to an individual, including any individual encompassed herein. Stem cells may be administered to the individual systemically and/or locally. Stem cells may be administered to the individual intravenously, intradiscally, epidurally, intrarectally, intra-omentally, intra-arterially, or a combination thereof. Stem cells may be administered prior to or simultaneously with the compositions and/or cellular therapies encompassed herein.

### V. Fibroblasts Cell Therapies

Certain embodiments of the present disclosure concern the administration of one or more cellular therapies, such as at least one fibroblasts therapy and/or stem cell therapy. A cellular therapy, such as a fibroblast cell therapy may comprise a therapeutically effective amount of cells, such as fibroblasts. The cellular therapy may comprise one or more other suitable compositions that allow for the generation, storage, and/or administration of the cellular therapy. Fibroblasts may be administered to the individual systemically and/or locally. Fibroblasts may be administered to the individual intravenously, intradiscally, epidurally, intrarectally, intra-omentally, intra-arterially, or a combination thereof. Fibroblasts may be administered prior to or simultaneously with the compositions and/or cellular therapies encompassed herein.

There are several methods known in the art for the generation of fibroblasts or obtaining them. In some embodiments, the fibroblasts are from omentum, bone marrow, placenta, peripheral blood, cord blood, Wharton's jelly, cerebral spinal fluid, cancer-associated, foreskin, skin, a combination thereof, or any other tissue sufficiently abundant in fibroblasts. The fibroblasts of the present disclosure may comprise fibroblasts selected from the group consisting of skin fibroblasts, hair follicle fibroblasts, adipose fibroblasts, bone marrow fibroblasts, umbilical cord blood fibroblasts, placental fibroblasts, omentum fibroblasts, ovarian tube fibroblasts, peripheral blood fibroblasts, and a combination thereof. In some embodiments, fibroblasts of the present disclosure are isolated from tissue comprising fibroblasts selected from the group consisting of skin fibroblasts, hair follicle fibroblasts, adipose fibroblasts, bone marrow fibroblasts, umbilical cord blood fibroblasts, placental fibroblasts, omentum fibroblasts, ovarian tube fibroblasts, peripheral blood fibroblasts, and a combination thereof.

In some embodiments, fibroblasts are generated according to protocols previously utilized for treatment of patients utilizing bone marrow-derived MSCs. Specifically, bone marrow is aspirated (10-30 mL) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2×10⁷ cells/ml. Subsequently the cells are centrifuged at approximately 900 g for approximately 30 min or a time period sufficient to achieve separation of mononuclear cells from debris and erythrocytes. The cells are then washed with PBS and plated at a density of approximately 1×10⁶ cells per ml in 175 cm² tissue culture flasks in DMEM with 10% FCS with flasks subsequently being loaded with a minimum of 30 million bone marrow mononuclear cells. The fibroblasts are allowed to adhere for 72 h followed by media changes every 3-4 days. Adherent cells are removed with 0.05% trypsin-EDTA and replated at a density of approximately 1×10⁶ per 175 cm².

Fibroblasts may be cultured in the presence of a liquid culture medium. Typically, the medium may comprise a basal medium formulation as known in the art. Many basal media formulations can be used to culture fibroblasts herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the fibroblasts cultured. In some embodiments, a culture medium formulation may be explants medium (CEM) which is composed of IMDM supplemented with 10% fetal bovine serum (FBS, Lonza), 100 U/ml penicillin G, 100 μg/ml streptomycin and 2 mmol/L L-glutamine (Sigma-Aldrich). Other embodiments may employ further basal media formulations, such as chosen from the ones above.

It is known that under certain conditions fibroblasts are capable of producing interleukin-1 and/or other inflammatory cytokines [106]. The disclosure concerns methods to manipulate fibroblasts to reduce and/or inhibit the production and/or secretion of inflammatory cytokines, such as by gene editing and/or exposure to one or more compositions that aid in the reduction or inhibition of the production and/or secretion of inflammatory cytokines. In certain embodiments, fibroblasts may be manipulated by gene editing of IL-1 and/or other inflammatory mediators, said inflammatory mediators are selected from a group comprising of interleukin-1, interleukin-6, interleukin-7, interleukin-9, interleukin-11, interleukin-12, interleukin-8, interleukin-15, interleukin-17, interleukin-18, interleukin-21, interleukin-23, interleukin-27, interleukin-33, interferon-gamma, TNF-alpha, HMGB-1 and a combination thereof, including in order to prevent expression and/or excretion of inflammatory cytokines by the fibroblasts after the fibroblasts are administered intradiscally to an individual.

Gene editing may comprise genomic manipulation, such as at least one genetic knockout by any method (including CRISPR systems, TALEN systems, recombination systems, or a combination thereof) and/or at least one transgenic insertion. Gene editing may comprise RNA interference, such as RNA interference that targets inflammatory cytokines encompassed herein. In some embodiments, gene editing comprises the use of a viral construct. In some embodiments, gene editing comprises the use of a non-viral construct.

In some embodiments of the disclosure, TNF-alpha and inflammatory mediators are suppressed in the disc, fibroblasts are pretreated with TNF-alpha in a manner to induce expression of growth factors and/or proliferation such as described in this following publication and incorporated by reference [107, 108]. In some embodiments, fibroblasts comprising at least one fibroblast cell therapy are exposed to TNF-alpha prior to administering the fibroblasts. In some embodiments, exposure to TNF-alpha prevents and/or reduces the expression and/or secretion of inflammatory cytokines including interleukin-1, interleukin-6, interleukin-7, interleukin-9, interleukin-11, interleukin-12, interleukin-8, interleukin-15, interleukin-17, interleukin-18, interleukin-21, interleukin-23, interleukin-27, interleukin-33, interferon-gamma, TNF-alpha, HMGB-1 and a combination thereof. In some embodiments, fibroblasts comprising at least one fibroblast cell therapy are exposed to at least one inhibitor of protein kinase C, cyclic nucleotide-dependent protein kinases, calmodulin-dependent protein kinases, the Na(+)-H+ antiport system, or a combination thereof prior to administering the fibroblasts. In some embodiments, exposure to at least one inhibitor of protein kinase C, cyclic nucleotide-dependent protein kinases, calmodulin-dependent protein kinases, the Na(+)-H+ antiport system, or a combination thereof prevents and/or reduces the expression and/or secretion of inflammatory cytokines including interleukin-1, interleukin-6, interleukin-7, interleukin-9, interleukin-11, interleukin-12, interleukin-8, interleukin-15, interleukin-17, interleukin-18, interleukin-21, interleukin-23, interleukin-27, interleukin-33, interferon-gamma, TNF-alpha, HMGB-1 and a combination thereof.

Certain embodiments of the present disclosure concern augmenting the efficacy of a cell therapy, such as a fibroblast cell therapy, for degenerative disc disease comprising augmenting receptivity of disc tissue for the cell therapy. In some embodiments, augmentation of the receptivity comprises reducing inflammation in the disc tissue. In some embodiments, fibroblasts, such as those comprising a fibroblast cell therapy, are cultured in a manner, including in conditions of hypoxia, to adapt the fibroblasts to the intradiscal environment of an individual.

### VI. References

1. Cohen, S. P., et al., A double-blind, placebo-controlled, dose-response pilot study evaluating intradiscal etanercept in patients with chronic discogenic low back pain or lumbosacral radiculopathy. Anesthesiology, 2007. 107(1): p. 99-105.
2. Tobinick, E. and S. Davoodifar, Efficacy of etanercept delivered by perispinal administration for chronic back and/or neck disc-related pain: a study of clinical observations in 143 patients. Curr Med Res Opin, 2004. 20(7): p. 1075-85.
3. Tobinick, E., Perispinal etanercept for neuroinflammatory disorders. Drug Discov Today, 2009. 14(3-4): p. 168-77.
4. Tobinick, E. L. and S. Britschgi-Davoodifar, Perispinal TNF-alpha inhibition for discogenic pain. Swiss Med Wkly, 2003. 133(11-12): p. 170-7.
5. Voitenok, N. N., et al., Induction of tumor necrosis factor synthesis in human monocytes treated by transcriptional inhibitors. Immunol Lett, 1989. 20(1): p. 77-82.
6. Evans, G. F. and S. H. Zuckerman, Pharmacologic modulation of TNF production by endotoxin stimulated macrophages: in vitro and in vivo effects of auranofin and other chrysotherapeutic compounds. Agents Actions, 1989. 26(3-4): p. 329-34.
7. Schade, U. F., et al., Lipoxygenase inhibitors suppress formation of tumor necrosis factor in vitro and in vivo. Biochem Biophys Res Commun, 1989. 159(2): p. 748-54.
8. Schade, U. F., R. Engel, and D. Jakobs, The role of lipoxygenases in endotoxin-induced cytokine production. Prog Clin Biol Res, 1991. 367: p. 73-82.
9. Schade, F. U., R. Engel, and D. Jakobs, Lipoxygenase inhibitors but not site specific 5-lipoxygenase blockers protect against endotoxic shock and inhibit production of tumor necrosis factor. Eicosanoids, 1992. 5 Suppl: p. S45-7.
10. Elekes, E., D. Jakobs, and F. U. Schade, Suppression of endotoxin mitogenicity of spleen cells by lipoxygenase inhibitors and its reversal by 13-hydroxyoctadecadienoic acid. FEMS Immunol Med Microbiol, 1993. 6(1): p. 13-20.
11. D'Souza, N. B., et al., Acute alcohol infusion suppresses endotoxin-induced serum tumor necrosis factor. Alcohol Clin Exp Res, 1989. 13(2): p. 295-8.
12. Bermudez, L. E., et al., Ethanol affects release of TNF and GM-CSF and membrane expression of TNF receptors by human macrophages. Lymphokine Cytokine Res, 1991. 10(5): p. 413-9.
13. Dubois, C. M., E. Bissonnette, and M. Rola-Pleszczynski, Asbestos fibers and silica particles stimulate rat alveolar macrophages to release tumor necrosis factor. Autoregulatory role of leukotriene B4. Am Rev Respir Dis, 1989. 139(5): p. 1257-64.
14. Essner, R., et al., IL-4 down-regulates IL-1 and TNF gene expression in human monocytes. J Immunol, 1989. 142(11): p. 3857-61.
15. Di Santo, E., et al., IL-13 inhibits TNF production but potentiates that of IL-6 in vivo and ex vivo in mice. J Immunol, 1997. 159(1): p. 379-82.
16. Stokes, D. C., et al., Polymyxin B prevents lipopolysaccharide-induced release of tumor necrosis factor-alpha from alveolar macrophages. J Infect Dis, 1989. 160(1): p. 52-7.
17. Pappo, I., et al., Polymyxin B reduces total parenteral nutrition-associated hepatic steatosis by its antibacterial activity and by blocking deleterious effects of lipopolysaccharide. JPEN J Parenter Enteral Nutr, 1992. 16(6): p. 529-32.
18. Greve, J. W., D. J. Gouma, and W. A. Buurman, Bile acids inhibit endotoxin-induced release of tumor necrosis factor by monocytes: an in vitro study. Hepatology, 1989. 10(4): p. 454-8.
19. Aderka, D., J. M. Le, and J. Vilcek, IL-6 inhibits lipopolysaccharide-induced tumor necrosis factor production in cultured human monocytes, U937 cells, and in mice. J Immunol, 1989. 143(11): p. 3517-23.
20. Greve, J. W., et al., Lactulose inhibits endotoxin induced tumour necrosis factor production by monocytes. An in vitro study. Gut, 1990. 31(2): p. 198-203.
21. Waage, A., M. Sorensen, and B. Stordal, Differential effect of oxpentifylline on tumour necrosis factor and interleukin-6 production. Lancet, 1990. 335(8688): p. 543.
22. Barton, B. E., et al., Cytokine inhibition by a novel steroid, mometasone furoate. Immunopharmacol Immunotoxicol, 1991. 13(3): p. 251-61.
23. Kutteh, W. H., W. E. Rainey, and B. R. Carr, Glucocorticoids inhibit lipopolysaccharide-induced production of tumor necrosis factor-alpha by human fetal Kupffer cells. J Clin Endocrinol Metab, 1991. 73(2): p. 296-301.
24. Allen, J. N., D. J. Herzyk, and M. D. Wewers, Colchicine has opposite effects on interleukin-1 beta and tumor necrosis factor-alpha production. Am J Physiol, 1991. 261(4 Pt 1): p. L315-21.
25. Jeong, J. Y. and D. M. Jue, Chloroquine inhibits processing of tumor necrosis factor in lipopolysaccharide-stimulated RAW 264.7 macrophages. J Immunol, 1997. 158(10): p. 4901-7.
26. Kawano, K., et al., FK 506 ameliorates normothermic liver ischemia in rats by suppressing production of tumor necrosis factor. Transpl Int, 1992. 5 Suppl 1: p. S665-9.
27. Kawano, K., et al., Evidence that FK506 alleviates ischemia/reperfusion injury to the rat liver: in vivo demonstration for suppression of TNF-α production in response to endotoxemia. Eur Surg Res, 1994. 26(2): p. 108-15.
28. Smith, C. S., et al., Cyclosporin A blocks induction of tumor necrosis factor-alpha in human B lymphocytes. Biochem Biophys Res Commun, 1994. 204(1): p. 383-90.
29. Molnar-Kimber, K. L., et al., Differential regulation of TNF-alpha and IL-1beta production from endotoxin stimulated human monocytes by phosphodiesterase inhibitors. Mediators Inflamm, 1992. 1(6): p. 411-7.
30. Wang, M. H., et al., Inhibition of endotoxin or lipid A-induced tumor necrosis factor production by synthetic lipid A partial structures in human peripheral blood mononuclear cells. Lymphokine Cytokine Res, 1992. 11(1): p. 23-31.
31. Molnar-Kimber, K., et al., Modulation of TNF alpha and IL-1 beta from endotoxin-stimulated monocytes by selective PDE isozyme inhibitors. Agents Actions, 1993. 39 Spec No: p. C77-9.
32. Giroir, B. P. and B. Beutler, Effect of amrinone on tumor necrosis factor production in endotoxic shock. Circ Shock, 1992. 36(3): p. 200-7.
33. Peristeris, P., et al., N-acetylcysteine and glutathione as inhibitors of tumor necrosis factor production. Cell Immunol, 1992. 140(2): p. 390-9.
34. Schreck, R., et al., Dithiocarbamates as potent inhibitors of nuclear factor kappa B activation in intact cells. J Exp Med, 1992. 175(5): p. 1181-94.
35. Thomassen, M. J., et al., Synthetic surfactant (Exosurf) inhibits endotoxin-stimulated cytokine secretion by human alveolar macrophages. Am J Respir Cell Mol Biol, 1992. 7(3): p. 257-60.
36. Danenberg, H. D., et al., Dehydroepiandrosterone protects mice from endotoxin toxicity and reduces tumor necrosis factor production. Antimicrob Agents Chemother, 1992. 36(10): p. 2275-9.
37. Zheng, Z. M., S. Specter, and H. Friedman, Serum proteins affect the inhibition by delta-tetrahydrocannabinol of tumor necrosis factor alpha production by mouse macrophages. Adv Exp Med Biol, 1993. 335: p. 89-93.
38. Fischer-Stenger, K., D. A. Dove Pettit, and G. A. Cabral, Delta 9-tetrahydrocannabinol inhibition of tumor necrosis factor-alpha: suppression of post-translational events. J Pharmacol Exp Ther, 1993. 267(3): p. 1558-65.
39. Monastra, G., et al., Phosphatidylserine, a putative inhibitor of tumor necrosis factor, prevents autoimmune demyelination. Neurology, 1993. 43(1): p. 153-63.
40. Ogata, M., et al., An antagonist of platelet-activating factor suppresses endotoxin-induced tumor necrosis factor and mortality in mice pretreated with carrageenan. Infect Immun, 1993. 61(2): p. 699-704.
41. Moreira, A. L., et al., Thalidomide exerts its inhibitory action on tumor necrosis factor alpha by enhancing mRNA degradation. J Exp Med, 1993. 177(6): p. 1675-80.
42. Tramontana, J. M., et al., Thalidomide treatment reduces tumor necrosis factor alpha production and enhances weight gain in patients with pulmonary tuberculosis. Mol Med, 1995. 1(4): p. 384-97.
43. Schmidt, H., et al., Thalidomide inhibits TNF response and increases survival following endotoxin injection in rats. J Surg Res, 1996. 63(1): p. 143-6.
44. Fantuzzi, G., et al., Inhibitors of cytochrome P450 suppress tumor necrosis factor production. Cell Immunol, 1993. 150(2): p. 417-24.
45. Shinji, H., K. S. Akagawa, and T. Yoshida, Cytochalasin D inhibits lipopolysaccharide-induced tumor necrosis factor production in macrophages. J Leukoc Biol, 1993. 54(4): p. 336-42.
46. Takenaka, I., et al., Ketamine suppresses endotoxin-induced tumor necrosis factor alpha production in mice. Anesthesiology, 1994. 80(2): p. 402-8.
47. Stevens, D. B., K. E. Gould, and R. H. Swanborg, Transforming growth factor-beta 1 inhibits tumor necrosis factor-alpha/lymphotoxin production and adoptive transfer of disease by effector cells of autoimmune encephalomyelitis. J Neuroimmunol, 1994. 51(1): p. 77-83.
48. Wang, P., et al., Interleukin (IL)-10 inhibits nuclear factor kappa B (NF kappa B) activation in human monocytes. IL-10 and IL-4 suppress cytokine synthesis by different mechanisms. J Biol Chem, 1995. 270(16): p. 9558-63.
49. Bernard, C., et al., Pentoxifylline selectivity inhibits tumor necrosis factor synthesis in the arterial wall. J Cardiovasc Pharmacol, 1995. 25 Suppl 2: p. S30-3.
50. Kaplan, J. M., et al., Effect of TNF alpha production inhibitors BRL 61063 and pentoxifylline on the response of rats to poly I:C. Toxicology, 1995. 95(1-3): p. 187-96.
51. Hotchkiss, R. S., et al., Calcium antagonists decrease plasma and tissue concentrations of tumor necrosis factor-alpha, interleukin-1 beta, and interleukin-1 alpha in a mouse model of endotoxin. Shock, 1995. 3(5): p. 337-42.
52. Chan, M. M., Inhibition of tumor necrosis factor by curcumin, a phytochemical. Biochem Pharmacol, 1995. 49(11): p. 1551-6.
53. Belkowski, S. M., et al., Inhibition of interleukin-1 and tumor necrosis factor-alpha synthesis following treatment of macrophages with the kappa opioid agonist U50, 488H. J Pharmacol Exp Ther, 1995. 273(3): p. 1491-6.
54. Sansoni, P., et al., Inhibition of antigen-presenting cell function by alendronate in vitro. J Bone Miner Res, 1995. 10(11): p. 1719-25.
55. Onai, N., et al., Inhibitory effects of bisbenzylisoquinoline alkaloids on induction of proinflammatory cytokines, interleukin-1 and tumor necrosis factor-alpha. Planta Med, 1995. 61(6): p. 497-501.
56. Ferrante, A., et al., Tetrandrine, a plant alkaloid, inhibits the production of tumour necrosis factor-alpha (cachectin) by human monocytes. Clin Exp Immunol, 1990. 80(2): p. 232-5.
57. Bissonnette, E. Y., J. A. Enciso, and A. D. Befus, Inhibitory effects of sulfasalazine and its metabolites on histamine release and TNF-alpha production by mast cells. J Immunol, 1996. 156(1): p. 218-23.
58. van der Poll, T., et al., Epinephrine inhibits tumor necrosis factor-alpha and potentiates interleukin 10 production during human endotoxemia. J Clin Invest, 1996. 97(3): p. 713-9.
59. Warr, G. A., et al., BMS-182123, a fungal metabolite that inhibits the production of TNF-alpha by macrophages and monocytes. J Antibiot (Tokyo), 1996. 49(3): p. 234-40.
60. Sajjadi, F. G., et al., Inhibition of TNF-alpha expression by adenosine: role of A3 adenosine receptors. J Immunol, 1996. 156(9): p. 3435-42.
61. Eigler, A., et al., Endogenous adenosine curtails lipopolysaccharide-stimulated tumour necrosis factor synthesis. Scand J Immunol, 1997. 45(2): p. 132-9.
62. Goto, M., et al., Inhibitory effect of E3330, a novel quinone derivative able to suppress tumor necrosis factor-alpha generation, on activation of nuclear factor-kappa B. Mol Pharmacol, 1996. 49(5): p. 860-73.
63. Li, Q., et al., Nicotine reduces TNF-alpha expression through a alpha7 nAChR/MyD88/NF-kB pathway in HBE16 airway epithelial cells. Cell Physiol Biochem, 2011. 27(5): p. 605-12.
64. Madretsma, G. S., et al., Nicotine inhibits the in vitro production of interleukin 2 and tumour necrosis factor-alpha by human mononuclear cells. Immunopharmacology, 1996. 35(1): p. 47-51.
65. Toungouz, M., C. Denys, and E. Dupont, Blockade of proliferation and tumor necrosis factor-alpha production occurring during mixed lymphocyte reaction by interferon-gamma-specific natural antibodies contained in intravenous immunoglobulins. Transplantation, 1996. 62(9): p. 1292-6.
66. Darville, T., L. B. Milligan, and K. K. Laffoon, Intravenous immunoglobulin inhibits staphylococcal toxin-induced human mononuclear phagocyte tumor necrosis factor alpha production. Infect Immun, 1997. 65(2): p. 366-72.
67. Benigni, F., et al., Cardiotrophin-1 inhibits tumor necrosis factor production in the heart and serum of lipopolysaccharide-treated mice and in vitro in mouse blood cells. Am J Pathol, 1996. 149(6): p. 1847-50.
68. Feng, Y., et al., Inhibition of LPS-induced TNF-alpha production by calcitonin gene-related peptide (CGRP) in cultured mouse peritoneal macrophages. Life Sci, 1997. 61(20): p. PL 281-7.
69. Qiu, H. B., et al., Effects of dexamethasone, ibuprofen, and ligustrazini on lipopolysaccharides-induced tumor necrosis factor alpha production. Zhongguo Yao Li Xue Bao, 1997. 18(1): p. 67-70.
70. Shohami, E., et al., Cytokine production in the brain following closed head injury: dexanabinol (HU-211) is a novel TNF-alpha inhibitor and an effective neuroprotectant. J Neuroimmunol, 1997. 72(2): p. 169-77.
71. Jones, A., et al., Inhibition of tumour necrosis factor production in endotoxin-stimulated human mononuclear leukocytes by the prostacyclin analogue iloprost: cellular mechanisms. Cytokine, 1997. 9(2): p. 119-25.
72. Murakami, K., et al., Activated protein C prevents LPS-induced pulmonary vascular injury by inhibiting cytokine production. Am J Physiol, 1997. 272(2 Pt 1): p. L197-202.
73. Thissen, J. P. and J. Verniers, Inhibition by interleukin-1 beta and tumor necrosis factor-alpha of the insulin-like growth factor I messenger ribonucleic acid response to growth hormone in rat hepatocyte primary culture. Endocrinology, 1997. 138(3): p. 1078-84.
74. Zhang, M., et al., Spermine inhibits proinflammatory cytokine synthesis in human mononuclear cells: a countenegulatory mechanism that restrains the immune response. J Exp Med, 1997. 185(10): p. 1759-68.
75. Yamamoto, N., et al., FR167653, a dual inhibitor of interleukin-1 and tumor necrosis factor-alpha, ameliorates endotoxin-induced shock. Eur J Pharmacol, 1997. 327(2-3): p. 169-74.
76. Solorzano, C. C., et al., A matrix metalloproteinase inhibitor prevents processing of tumor necrosis factor alpha (TNF alpha) and abrogates endotoxin-induced lethality. Shock, 1997. 7(6): p. 427-31.
77. Deshpande, R., et al., Estradiol down-regulates LPS-induced cytokine production and NFkB activation in murine macrophages. Am J Reprod Immunol, 1997. 38(1): p. 46-54.
78. Shackelford, R. E., et al., Aspirin inhibits tumor necrosis factoralpha gene expression in murine tissue macrophages. Mol Pharmacol, 1997. 52(3): p. 421-9.
79. Matsumori, A., et al., Amiodarone inhibits production of tumor necrosis factor-alpha by human mononuclear cells: a possible mechanism for its effect in heart failure. Circulation, 1997. 96(5): p. 1386-9.
80. Kettelhut, I. C., W. Fiers, and A. L. Goldberg, The toxic effects of tumor necrosis factor in vivo and their prevention by cyclooxygenase inhibitors. Proc Natl Acad Sci USA, 1987. 84(12): p. 4273-7.
81. Rand, T. H., et al., Nedocromil sodium and cromolyn (sodium cromoglycate) selectively inhibit antibody-dependent granulocyte-mediated cytotoxicity. Int Arch Allergy Appl Immunol, 1988. 87(2): p. 151-8.
82. Lin, Y., P. G. Case, and P. Q. Patek, Inhibition of tumour necrosis factor and natural cytotoxic cell lytic activities by a spleen cell-elaborated factor. Immunology, 1988. 63(4): p. 663-8.
83. Sullivan, G. W., et al., Inhibition of the inflammatory action of interleukin-1 and tumor necrosis factor (alpha) on neutrophil function by pentoxifylline. Infect Immun, 1988. 56(7): p. 1722-9.
84. Gibson, R. L., et al., Group B streptococcus induces tumor necrosis factor in neonatal piglets. Effect of the tumor necrosis factor inhibitor pentoxifylline on hemodynamics and gas exchange. Am Rev Respir Dis, 1991. 143(3): p. 598-604.
85. Kozaki, K., et al., Pentoxifylline inhibits production of superoxide anion and tumor necrosis factor by Kupffer cells in rat liver preservation. Transplant Proc, 1993. 25(6): p. 3025-6.
86. Seckinger, P., et al., Tumor necrosis factor inhibitor: purification, NH2-terminal amino acid sequence and evidence for anti-inflammatory and immunomodulatory activities. Eur J Immunol, 1990. 20(5): p. 1167-74.
87. Kilbourn, R. G., et al., NG-methyl-L-arginine inhibits tumor necrosis factor-induced hypotension: implications for the involvement of nitric oxide. Proc Natl Acad Sci USA, 1990. 87(9): p. 3629-32.
88. Mayoral, J. L. and D. L. Dunn, Cross-reactive murine monoclonal antibodies directed against the core/lipid A region of endotoxin inhibit production of tumor necrosis factor. J Surg Res, 1990. 49(4): p. 287-92.
89. Mukaida, N., et al., Dexamethasone inhibits the induction of monocyte chemotactic-activating factor production by IL-1 or tumor necrosis factor. J Immunol, 1991. 146(4): p. 1212-5.
90. Gadina, M., et al., Protective effect of chlorpromazine on endotoxin toxicity and TNF production in glucocorticoid-sensitive and glucocorticoid-resistant models of endotoxic shock. J Exp Med, 1991. 173(6): p. 1305-10.
91. LaMarre, J., et al., Cytokine binding and clearance properties of proteinase-activated alpha 2-macroglobulins. Lab Invest, 1991. 65(1): p. 3-14.
92. Shainkin-Kestenbaum, R., et al., Acute phase protein, serum amyloid A, inhibits IL-1- and TNF-induced fever and hypothalamic PGE2 in mice. Scand J Immunol, 1991. 34(2): p. 179-83.
93. van Kessel, K. P., J. A. van Strijp, and J. Verhoef, Inactivation of recombinant human tumor necrosis factor-alpha by proteolytic enzymes released from stimulated human neutrophils. J Immunol, 1991. 147(11): p. 3862-8.
94. Bagby, G. J., et al., Attenuation of glucose metabolic changes resulting from TNF-alpha administration by adrenergic blockade. Am J Physiol, 1992. 262(4 Pt 2): p. R628-35.
95. Ogata, M., et al., Protective effects of a leukotriene inhibitor and a leukotriene antagonist on endotoxin-induced mortality in carrageenan-pretreated mice. Infect Immun, 1992. 60(6): p. 2432-7.
96. Parry, E. W., Cycloheximide or nordihydroguaiaretic acid protects mice against the lethal and hepatocytolytic effects of a combined challenge with D-galactosamine and bacterial endotoxin. J Comp Pathol, 1993. 108(2): p. 185-90.
97. van Hinsbergh, V. W., et al., Genistein reduces tumor necrosis factor alpha-induced plasminogen activator inhibitor-1 transcription but not urokinase expression in human endothelial cells. Blood, 1994. 84(9): p. 2984-91.
98. Taniguchi, S., et al., Butylated hydroxyanisole blocks the inhibitory effects of tumor necrosis factor-alpha on collagen production in human dermal fibroblasts. J Dermatol Sci, 1996. 12(1): p. 44-9.
99. Cohen, P. S., et al., CNI-1493 inhibits monocyte/macrophage tumor necrosis factor by suppression of translation efficiency. Proc Natl Acad Sci USA, 1996. 93(9): p. 3967-71.
100. Zhang, J. P., et al., Inhibitory effect of quercetin on tumor necrosis factor and interleukin-1 beta pro-osteoclastic activities. Zhongguo Yao Li Xue Bao, 1996. 17(3): p. 261-3.
101. Murakami, K., et al., Gabexate mesilate, a synthetic protease inhibitor, attenuates endotoxin-induced pulmonary vascular injury by inhibiting tumor necrosis factor production by monocytes. Crit Care Med, 1996. 24(6): p. 1047-53.
102. Murakami, K., et al., A novel platelet activating factor antagonist, SM-12502, attenuates endotoxin-induced disseminated intravascular coagulation and acute pulmonary vascular injury by inhibiting TNF production in rats. Thromb Haemost, 1996. 75(6): p. 965-70.
103. Suzuki, K., et al., Monoclonal nonspecific suppressor factor beta (MNSF beta) inhibits the production of TNF-alpha by lipopolysaccharide-activated macrophages. Immunobiology, 1996. 195(2): p. 187-98.
104. Munoz, C., et al., Pyrrolidine dithiocarbamate inhibits the production of interleukin-6, interleukin-8, and granulocyte-macrophage colony-stimulating factor by human endothelial cells in response to inflammatory mediators: modulation of NF-kappa B and AP-1 transcription factors activity. Blood, 1996. 88(9): p. 3482-90.
105. Hill, G. E. and R. A. Robbins, Aprotinin but not tranexamic acid inhibits cytokine-induced inducible nitric oxide synthase expression. Anesth Analg, 1997. 84(6): p. 1198-202.
106. Yamato, K., Z. el-Hajjaoui, and H. P. Koeffler, Regulation of levels of IL-1 mRNA in human fibroblasts. J Cell Physiol, 1989. 139(3): p. 610-6.
107. Zucali, J. R., C. Morse, and C. A. Dinarello, The role of protein kinase C in interleukin 1 and tumor necrosis factor alpha induction of fibroblasts to produce and release granulocyte-macrophage colony-stimulating activity. Exp Hematol, 1990. 18(8): p. 888-92.
108. Hori, T., et al., Prostaglandins antagonize fibroblast proliferation stimulated by tumor necrosis factor. Biochem Biophys Res Commun, 1991. 174(2): p. 758-66.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of increasing the efficacy of a fibroblast cell therapy for a degenerative disc disease, comprising the steps of:
a. administering
i. at least one anti-inflammatory composition,
ii. exosomes and/or apoptotic bodies,
iii. stem cells, or
iv. a combination thereof; and
b. administering the fibroblast cell therapy.

2. The method of claim 1, wherein the anti-inflammatory composition comprises a composition that inhibits and/or reduces TNF-alpha.

3. The method of claim 2, wherein the composition that inhibits and/or reduces TNF-alpha comprises a composition selected from the group consisting of etanercept, methylprednisolone, cycloheximide, auranofin, sodium aurothiomalate, triethyl gold phosphine Ethanol, leukotriene B4, interleukin-4, interleukin-13, polymyxin B, bile acids, interleukin-6, lactulose, oxpentifylline, mometasone, glucocorticoids, colchicine, chloroquine, FK-506, berberine, resveratrol, pterostilbene, vitamin A, vitamin C, cyclosporine, phosphodiesterase inhibitors such as vinpocetine, milrinone, CI-930, rolipram, nitroquazone, zaprinast, synthetic lipid A, amrinone, N-acetylcysteine, dithiocarbamates and metal chelators, exosurf synthetic surfactant, dehydroepiandrosterone, delta-tetrahydrocannabinol, phosphatidylserine, TCV-309, PAF antagonist, thalidomide, cytochrome p450 inhibitors such as Metyrapone and SKF525A, cytochalasin D, ketamine, TGF-beta, interleukin-10, pentoxifylline, BRL 61,063, calcium antagonists such as dantrolene, azumolene, and diltiazem, curcumin, kappa-selective opioid agoinst U50,488H (trans-3,4-dichloro-N-methyl-N-[7-(1-pyrrolidinyl)cyclohexyl]benzene-acetamide methanesulfonate), alendronate, alkaloids such as fangchinoline and isotetrandrine, plant alkaloids such as tetrandrine, sulfasalazine, epinephrine, BMS-182123, adenosine, E3330, nicotine, IVIG, cardiotrophin-1, KB-R7785, CGRP, ligustrazine, dexanabinol, iloprost, activated protein C, growth hormone, spermine, FR-167653, gm-6001, estradiol, aspirin, amiodarone, melatonin, and a combination thereof.

4. The method of claim 2, wherein the composition that inhibits and/or reduces TNF-alpha comprises melatonin.

5. The method of claim 1, wherein the anti-inflammatory composition comprises a composition that suppresses responsiveness of cells in the individual to activation of one or more TNF-alpha receptors.

6. The method of claim 1, wherein the anti-inflammatory composition comprises a composition that inhibits activity of TNF-alpha.

7. The method of claim 6, wherein the composition that inhibits activity of TNF-alpha comprises a composition selected from the group consisting of ibuprofen, indomethacin, nedocromil sodium, cromolyn (sodium cromoglycate), spleen derived factors, pentoxifylline, the 30 kDa TNF-alpha inhibitor, NG-methyl-L-arginine, antibodies directed against the core/lipid A, dexamethasone, chlorpromazine, activated alpha 2 macroglobulin, serum amyloid A protein, neutrophil derived proteolytic enzymes, phentolamine, propranolol, leukotriene inhibitors, nordihydroguaiaretic acid, genistein, butylated hydroxyanisole, CNI-1493, quercetin, gabexate mesylate, SM-12502, monoclonal nonspecific suppressor factor (MNSF), pyrrolidine dithiocarbamate (PDTC), aprotinin, and a combination thereof.

8. The method of claim 1, wherein the stem cells are modified.

9. The method of claim 8, wherein the stem cells are exposed to 1% oxygen for 24-48 hours.

10. The method of any one of claims 1-9, wherein the fibroblasts comprising the fibroblast cell therapy are modified to prevent or reduce expression or secretion of one or more inflammatory cytokines.

11. The method of claim 10, wherein the fibroblasts comprising the fibroblast cell therapy are modified to prevent or reduce the expression or secretion of interleukin-1, interleukin-6, interleukin-7, interleukin-9, interleukin-11, interleukin-12, interleukin-8, interleukin-15, interleukin-17, interleukin-18, interleukin-21, interleukin-23, interleukin-27, interleukin-33, interferon-gamma, TNF-alpha, HMGB-1, or a combination thereof.

12. The method of any one of claims 1-11, wherein the fibroblasts comprising the fibroblast cell therapy are contacted with TNF-alpha to prevent or reduce expression or secretion of one or more inflammatory cytokines.

13. The method of any one of claims 1-12, wherein the fibroblasts comprising the fibroblast cell therapy are exposed with TNF-alpha to prevent or reduce expression or secretion of IL-1, or a combination thereof.

14. The method of any one of claims 1-13, wherein the fibroblasts comprising the fibroblast cell therapy are cultured in a manner to adapt the fibroblasts to the intradiscal environment of the individual.

15. The method of any one of claims 1-14, wherein fibroblasts comprising the fibroblast cell therapy are cultured in conditions of hypoxia.

16. The method of any one of claims 1-15, wherein the fibroblasts comprising the fibroblast cell therapy are isolated from tissue comprising fibroblasts selected from the group consisting of skin fibroblasts, hair follicle fibroblasts, adipose fibroblasts, bone marrow fibroblasts, umbilical cord blood fibroblasts, placental fibroblasts, omentum fibroblasts, ovarian tube fibroblasts, peripheral blood fibroblasts, and a combination thereof.

17. The method of any one of claims 1-16, wherein the administering of step a) and/or step b) is in a disc of an individual.

18. The method of claim 17, wherein the disc comprises nucleus pulposus.

19. The method of either claim 17 or 18, wherein the administering of at least one anti-inflammatory composition and/or exosomes reduces inflammation in the disc.

20. The method of claim 19, wherein the inflammation in the disc is associated with an aspect selected from the group consisting of: the ERK2 pathway, the JAK/STAT pathway, the NF-kappa B pathway, perispinal neutrophils, perispinal monocytes, inflammatory cytokines, and a combination thereof.

21. The method of claim 20, wherein the inflammatory cytokines induce STAT3 activation in cells of hematopoietic origin.

22. The method of claim 21, wherein the cells of hematopoietic origin are monocytes.

23. The method of claim 20, wherein the inflammatory cytokines activate at least one inhibitor of kappa B kinase (IkB kinase)

24. The method of claim 20, wherein the inflammatory cytokines activate NF-kappa B.

25. The method of any one of claims 20-24, wherein the inflammatory cytokines are selected from the group consisting of interleukin-1, interleukin-6, interleukin-7, interleukin-9, interleukin-11, interleukin-12, interleukin-8, interleukin-15, interleukin-17, interleukin-18, interleukin-21, interleukin-23, interleukin-27, interleukin-33, interferon-gamma, TNF-alpha, HMGB-1, and a combination thereof.
